(19) United States
(12) Patent Application Publication
(10) Pub. No.: US 2022/0218877 A1
(43) Pub. Date: Jul. 14, 2022

(54) CANNABINOID POLYMERIC COMPOSITIONS

(71) Applicant: CANNASSURE LTD, Ashdod (IL)

(72) Inventors: Hadel ONALLAH SAAD, Um Al Fahem (IL); Shimon AMSELEM, Rehovot (IL); Nir PELES, Tel Aviv (IL)

(73) Assignee: CANNASSURE LTD, Ashdod (IL)

(21) Appl. No.: 17/604,420

(22) PCT Filed: Apr. 16, 2020
(86) PCT No.: PCT/IL2020/050449

§ 371 Date: Oct. 17, 2021

Related U.S. Application Data
(60) Provisional application No. 62/958,757, filed on Jan. 9, 2020.
(60) Provisional application No. 62/835,511, filed on Apr. 18, 2019.

(51) Int. Cl. A61L 27/54; A61K 31/352; A61K 31/05; A61F 6/14

(52) U.S. Cl. CPC A61L 27/54; A61K 31/352; A61L 31/08; A61F 6/14; A61K 31/05

(57) ABSTRACT

Described herein is a device, for insertion into a lumen of a human, the device comprising at least one cannabinoid. Optionally, the device is configured for insertion into a uterus of a female patient. The device is configured to gradually release the cannabinoid to the device's surroundings over time. Additionally, described herein are methods for treatment of a disease comprising administering to a patient in need thereof, a pharmaceutically acceptable amount of a cannabinoid via an intrauterine route. Optionally, the disease is endometrial cancer, uterine sarcoma, cervical cancer, Abnormal Pap Smears, Abnormal Uterine Bleeding, Endometriosis, Fibroids, Menopause, Ovarian Masses, Pelvic Pain, Polycystic Ovarian Syndrome, Sexually Transmitted Disease, Urinary Tract Infections, Vaginitis or ovarian cancer. Optionally, the disease is Lynch syndrome.

### PREVIOUS APPLICATIONS

This application claims the benefit under 35 U.S.C. § 119(e) of U.S. Provisional Patent Application No. 62/835,511 filed Apr. 18, 2019 and of U.S. Provisional Patent Application No. 62/958,757 filed Jan. 9, 2020, the contents of which are incorporated herein by reference in their entirety.

### FIELD

Provided herein are devices and compositions for treating conditions comprising cannabinoids, and methods for treatment using such devices.

### BACKGROUND

A drug-eluting stent or DES is a medical device, configured to fit within a lumen and optionally to maintain the lumen open within a biological organism. DESs may be formed of plastic or metal. DESs may also be formed to have a collapsed formation and an expanded formation. A DES is typically introduced into an organism in a collapsed formation, then upon reaching its proper destination, is transformed to have an expanded formation wherein the DES is secured within the organism.

An intrauterine device is a device for introduction into the uterus of a human. The device is configured to be placed in the uterus and to remain in the uterus for an extended period of time, preferably between 1 month and 10 years.

Both DESs and intrauterine devices may be coated with a pharmaceutically acceptable coating configured to release an active ingredient to the surroundings of the device.

### SUMMARY

Described herein is a device, for insertion into a lumen of a human, the device comprising at least one cannabinoid. Optionally, the device is configured for insertion into a uterus of a female patient, the device comprising at least one cannabinoid. The device is configured to gradually release the cannabinoid to the device's surroundings over time.

Additionally, described herein are methods for treatment of a disease comprising administering to a patient in need thereof, a pharmaceutically acceptable amount of a cannabinoid via an intrauterine route. Optionally, the disease is endometrial cancer, uterine sarcoma, cervical cancer, Abnormal Pap Smears, Abnormal Uterine Bleeding, Endometriosis, Fibroids, Menopause, Ovarian Masses, Pelvic Pain, Polycystic Ovarian Syndrome, Sexually Transmitted Disease, Urinary Tract Infections, Vaginitis or ovarian cancer. Optionally, the disease is Lynch syndrome.

The foregoing and other objects, features, and advantages will become more apparent from the following detailed description, which proceeds with reference to the accompanying figures.

### DETAILED DESCRIPTION

### Terms

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. The term “comprises” means “includes.” The abbreviation, “e.g.” is derived from the Latin exempli gratia and is used herein to indicate a non-limiting example. Thus, the abbreviation “e.g.” is synonymous with the term “for example.”

In case of conflict, the present specification, including explanations of terms, will control. In addition, all the materials, methods, and examples are illustrative and not intended to be limiting.

Biodegradable: able to break down in the body, for example, in the uterus, from solid form to non-solid form.
Cannabinoid: a compound active on the cannabinoid receptor in a human. Preferably, a phytocannabinoid. A cannabinoid may be produced synthetically, for example, through a chemical synthetic process or by using a biological organism such as a yeast or a bacteria modified to produce the cannabinoid. Alternatively, a cannabinoid may originate from a cannabis plant. A cannabinoid may be isolated, in pure form, or in combination with other cannabinoids.
Cannabis: a plant from the family Cannabaceae, optionally cannabis sativa, indica and ruderalis. Preferably a plant comprising a cannabinoid.
CBD: cannabidiol. A cannabinoid having the structure:

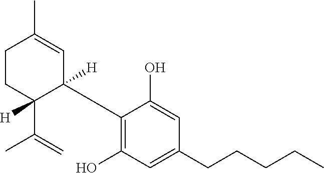 [SMILES: C=C(C)[C@@H]1CCC(C)=C[C@H]1Cc1c(O)cc(CCCCC)cc1O]

Chitosan: a mucoadhesive, linear polysaccharide derived from chitin obtained from crustacean shells. The interaction between cationic amino groups on chitosan and anionic moieties such as sialic and sulfonic acids on the mucus layer is responsible for its mucoadhesiveness. In addition, chitosan enhances epithelial permeability through the opening of tight junctions between epithelial cells.
Drug-Eluting Stent (DES): A metallic or plastic stent coated with a drug and preferably a polymer, configured to fit within a lumen and optionally to maintain the lumen open within a biological organism.
Endometrial Cancer: Endometrial cancer is a common type of cancer which affects only women, originating in the lining of the uterus. Endometrial cancer can be treated by radiation therapy, chemotherapy, hysterectomy or hormone therapy.
Intrauterine Device: A device for introduction into the uterus of a human. The device is configured to be placed in the uterus and to remain in the uterus for an extended period of time, preferably between 1 month and 10 years.
Lumen: the space surrounded by a tubular structure. Exemplary lumen include: a uterus, an artery and an intestine.
Lynch Syndrome: Although there are a number of risk factors increasing chances of endometrial cancer, one of the risk factors is a genetic disorder known as Lynch syndrome. Lynch syndrome sufferers are at high risk of colorectal cancer and endometrial cancer, in particular endometrial carcinoma.
Polycaprolactone: a biodegradable polymer (polyester) with the name (1,7)-Polyoxepan-2-one, also known as PCL. It undergoes hydrolysis in the presence of water.
Poly(lactic-co-glycolic acid): a biodegradable copolymer otherwise known as PLGA, is an aliphatic polyester comprising monomers of glycolic acid and lactic acid with ester linkages. PLGA undergoes hydrolysis in the presence of water.
THC: tetrahydrocannabinol. A cannabinoid having the structure:

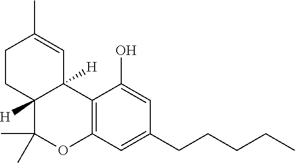 [SMILES: CCCCCc1cc(O)c2c(c1)OC(C)(C)[C@@H]1CCC(C)=C[C@@H]21]

Vitamin E TPGS: also known as d-α-tocopherol polyethylene glycol 1000 succinate. It is a water-soluble derivative of the natural form of vitamin E, d-α-tocopherol. It is produced by the esterification of crystalline d-α-tocopheryl succinate by polyethylene glycol 1000. Used as an emulsifier. It has an amphiphilic molecular structure with a polar hydrophilic head (consisting of polyethylene glycol) and a non-polar lipophilic tail (consisting of the phytyl chain of d-α-tocopherol). This makes it soluble in both water and oil.

Overview of Several Embodiments

Novel devices comprising a composition, the composition comprising at least one cannabinoid, in a controlled release form, configured to release the cannabinoid for a period of between 1 month and 5 years, or between 3 months and 5 years, are described herein. Methods to treat disease, including endometrial cancer, are also described herein.

According to an embodiment, at least one cannabinoid is present in a biodegradable polymer matrix configured to release the cannabinoid in a controlled fashion once implanted within the lumen. The matrix may be introduced into a cavity of a DES or an IUD. Alternatively, the matrix may coat a surface of the DES or the IUD.

Optionally, the matrix may further comprise a surfactant.

Optionally the matrix may further comprise a mucoadhesive agent. The mucoadhesive agent is preferably positively charged to allow enhanced electrostatic interaction with the anionic sialic and sulfonic acids moieties on the mucus layers thus improving the adhesiveness of an entrapped pharmacologically active compound such as a cannabinoid, to adhere to the mucosal surface. Preferably the mucoadhesive agent is chitosan known to enhance epithelial permeability through the opening of tight junctions between epithelial cells thus further improving the penetration of an entrapped pharmacologically active compound such as a cannabinoid.

Optionally, the composition may further comprise an organic solvent to dissolve the cannabinoid.

According to an embodiment, the cannabinoid present in the biodegradable polymer matrix is selected from the group consisting of cannabidiol (CBD) and tetrahydrocannabinol (THC). The biodegradable polymer matrix may comprise two cannabinoids in combination. The two cannabinoids may be CBD and THC. Optionally, the amount of THC in the biodegradable polymer matrix relative to total cannabinoid content is between 0.1% and 1%. The amount may be 0.2%

Other cannabinoids maybe but not limited to : cannabigerol (CBG), cannabigerolic acid (CBGA), cannabigerol monomethyl ether (CBGM), cannabichromene (CBC), cannabichromanone (CBCN), cannabichromenic acid (CBCA), cannabivarichromene (CBCV), cannabichromevarinic acid (CBCVA), isotetrahydrocannabinol (iso-THC), cannabinol (CBN), cannabinolic acid (CBNA), cannabinol methyl ether (CBNM), cannabinol C₄ (CBN-C₄), cannabinol C₂ (CBN-C₂), cannabinol C₁ (CBN-C₁), cannabinodiol (CBND), cannabielsoin (CBE), cannabielsoic acid A (CBEA-A), Cannabielsoic acid B (CBEA-B), cannabicyclol (CBL), cannabicycloic acid (CBLA), cannabicyclovarin (CBLV), cannabitriol (CBT), cannabitriolvarin (CBTV), ethoxy-cannabitriolvarin (CBTVE), cannabivarin (CBV), cannabinodivarin (CBVD), tetrahydrocannabivarin (THCV), cannabidivarin (CBDV), cannabigerovarin (CBGV), cannabigerovarinic acid (CBGVA), cannabifuran (CBF), dehydrocannabifuran DCBF, cannabirispol (CBR).

Cannabinoids are lipophilic substances with very poor water solubility. Cannabinoids such as THC, CBD, cannabinol, and their metabolites, are highly hydrophobic, lipid soluble compounds and can be dissolved in aqueous solutions only in the range of a few micrograms/ml or less, depending upon the conditions.

According to an embodiment, a DES or IUD comprises a biodegradable polymer, the polymer comprising between 1 mg and 100 mg of total cannabinoid.

The biodegradable polymer matrix may comprise cannabinoids in an amount of between 2% by weight, and 50% by weight, preferably between 20% and 50%.

Manufacture of Polymeric Matrix for use in Coating a DES or IUD

Biodegradable polymer matrix for use as a coating on a DES or on an IUD or within a DES or IUD may be prepared using the following procedure:

1. Dissolving a cannabinoid in an organic solvent.

2. Dissolving the mucoadhesive agent in organic, cannabinoid-containing solution.

3. Heating the biodegradable polymer, preferably polyester block copolymer, until it melts, in the presence of a surfactant.

4. Mixing the molten polymer with the organic, cannabinoid-containing solution to obtain a homogenous mixture.

5. Evaporating the solvents.

6. Cooling.

7. Combining biodegradable polymer matrix with the DES or IUD.

Organic solvents may be used to dissolve CBD and THC during the preparation of the biodegradable polymer matrix. Most of the solvent is removed by evaporation to complete dryness but some residual amounts of solvents may remain as excipients in the final composition and may act as a penetration enhancer through mucosal tissues. Optionally, the organic solvent used in the process may be selected from the group consisting of: ethanol, methanol, tertiary butanol, dimethyl sulfoxide, ethyl lactate, acetonitrile, and 2-(2-Ethoxyethoxy)ethanol.

Alternative methods for preparation of polymeric matrix for use in IUDs can be used. There are many different processing techniques such as solvent casting, particulate leaching, membrane lamination, freeze drying, phase separation, fiber bonding, electrospinning, melt based technologies, gas foaming, rapid prototyping, etc. [1). S. Ucar, P. Yilgor, V. Hasirci, and N. Hasirci, J. Appl. Polm. Sci., 130, 3759 (2013) .2). G. Vozzi, C. Flaim, A. Ahluwalia, and S. Bhatia, Biomaterials, 24, 2533 (2003).3). N. E. Vrana, A. Elsheikh, N. Builles, O. Damour, and V.Hasirci, Biomaterials, 28, 4303 (2007).4). Y. Zhang, J. R. Venugopal, A. El-Turki, S. Ramakrishna, and C. T. Lim, Biomaterials, 29, 4314 (2008).5). P. Yilgor, R. A. Sousa, R. L. Reis, N. Hasirci, and V.Hasirci, Macromol. Symp., 269, 92 (2008).6). D. Yucel, G. T. Kose, and V. Hasirci, Biomaterials, 31,1596 (2010)]. As a result of these processing techniques scaffolds in the forms of meshes, fibers, sponges, and foams can be obtained. Most techniques include the application of heat and/or pressure to the polymer or dissolving it in an organic solvent to mold the material into its desired shape.

The polymers to be used in the composition are biodegradable polyester block copolymers of prolonged release profile. Exemplary polymers which may be used include PLGA and PCL. Optionally, ethyl vinyl acetate (EVA) may be used. In addition to imparting controlled release properties, the polymeric matrix allows molding of the formulation into or onto the DES or IUD.

Various types of PLGA may be used in the biodegradable polymer matrix. Depending on the ratio of lactide to glycolide used for the polymerization, different forms of PLGA are available: these are usually identified regarding the molar ratio of the monomers used (e.g. PLGA 75:25 identifies a copolymer whose composition is 75% lactic acid and 25% glycolic acid). PLGA in ratios of polylactic to polyglycolic of 50:50, 70:30, 75:25, 82:18, and 85:15 can be used.

The crystallinity of PLGAs will vary from fully amorphous to fully crystalline depending on block structure and molar ratio. PLGAs typically show a glass transition temperature in the range of 40-60° C. PLGA can be dissolved by a wide range of solvents, depending on composition.

Additional polymers used to form the biodegradable polymer matrix may be polymers or copolymers comprising one or more of the monomers: L-lactide, ε-caprolactone, trimethylene carbonate, glycolide, D,L lactide, dioxanone.

The total amount of biodegradable copolymer to be added to the formulation is preferably within the range of 30-50% w/w (weight per total weight) of the final biodegradable polymer matrix.

The surfactants which may be used in the manufacture of the biodegradable polymer matrix composition are aimed to facilitate release of the drug from the biodegradable polymer matrix. Optionally, the surfactant used may be a nonionic surfactant selected from the group consisting of: vitamin E TPGS, polyethoxylated castor oil, polyethoxylated hydrogenated castor oil, polysorbates, sorbitan monooleate, poloxamers, macrogolglycerides linoleates, and mono- and di-fatty acid esters. Preferably, the surfactant is Vitamin E TPGS or polyethoxylated hydrogenated castor oil. The polyethoxylated hydrogenated castor oil may be polyoxyl 40 hydrogenated castor oil.

The amount of surfactant in the biodegradable polymer matrix composition may be in the range of 5-10% w/w.

As mentioned above, a positively charged mucoadhesive agent like chitosan will allow electrostatic interaction with to the anionic sialic and sulfonic acids moieties on the mucus layers thus improving the mucoadhesiveness of an entrapped pharmacologically active compound such as a cannabinoid improving its adherence to the mucosal surface. Mucins are a component of the vaginal/cervical mucosa. Mucins have an isoelectric point around 2-3 thus being negatively charged at vaginal/cervical pH. A positively charged mucoadhesive agent such as chitosan may allow the establishment of electrostatic bonding between the mucin and the positively-charged coated biodegradable polymer matrix comprising a cannabinoid.

Optionally, the mucoadhesive agent may be chitosan, or a chitosan derivative. The chitosan derivative may be 5-methyl-pyrrolidinone-chitosan (MPCS). The chitosan derivative may be a thiomer derivative of chitosan, which can be made by immobilization of thiol groups on chitosan. A degree of modification of 25-250 micromol thiol groups per gram chitosan leads to improvement in mucoadhesive properties. Thiomers are capable of forming intra- and interchain disulfide bonds within the polymeric network leading to strongly improved cohesive properties and the stability of drug delivery systems such as matrix tablets or rods can also be improved with thiomers. Chitosan-thioglycolic acid conjugates may also be used as a mucoadhesive agent.

The amount of mucoadhesive agent in biodegradable polymer matrix may be preferably in the range of 1-10% w/w.

Intrauterine Devices (IUD):

According to an embodiment, an IUD used to deliver a composition to the uterus of a patient in need thereof comprises: a stabilizing unit and reservoir. Optionally, the IUD comprises a removal member. The IUD may be T shaped.

The stabilizing unit may comprise an arm or plurality of arms which ensure proper positioning of the IUD within the uterus. The reservoir may comprise a porous receptacle having a hollow. The hollow may be filled with biodegradable polymer matrix comprising a cannabinoid, as described above. The IUD hollow may allow fluids to flow from the uterus to within the hollow of the reservoir. Optionally, the hollow may be cylindrical. The hollow covered with a membrane. The membrane may comprise silicone. The removal may comprise a cord which can be pulled to begin collapse of the IDU and removal of the IUD from the uterus.

The IUD may comprise a radiopaque substance to allow for visualization via radiography techniques. The radiopaque substance may be barium sulfate.

The IUD may be packaged with an inserter to compress the plurality of arms for insertion of the IUD into place. Upon insertion, the inserter may be removed from the vaginal cavity and discarded.

Other forms of IUD may include a vaginal ring. A vaginal ring may be formed from a flexible polymer. A vaginal ring may be inserted by folding or squeezing the ring, then introducing the ring, manually, into the vagina.

Other forms of IUD may include an intrauterine ball. The intrauterine ball may comprise an elongate conformable member comprising shape memory alloy, adapted to be pushed out of a sleeve into a uterine cavity. Upon entering the uterine cavity, the elongate conformable member may take a ball-like shape. A portion of the intrauterine ball may be coated with or otherwise incorporated with a cannabinoid-containing biodegradable matrix for release of cannabinoid in the uterus. Methods for manufacture of intrauterine ball devices are described in U.S. Pat. No. 9,750,634, incorporated herein by reference.

DESs:

According to an embodiment, the DES is selected from the group consisting of: a coronary DES, a vascular DES, a biliary DES, a bladder DES, a duodenal DES, a pancreatic DES, and a colonic DES. The DES may be made of a metal, such as a flexible metal, including nitinol.

Coronary DESs are stents used to keep arteries supplying blood to the heart open. vascular DESs are used for arteries of the periphery. Coronary DESs may be used for treatment of coronary heart disease, angina, and acute myocardial infarction

Biliary DESs are commonly used to open and alleviate pain caused by obstructions. Obstructions in the biliary tract can result from gallstones, injury, congenital malformations and biliary system related malignancies such as bile duct cancer and pancreatic cancer. A cannabinoid eluting biliary DES maybe used to treat pain, inflammation and cancer growth associated with biliary duct obstruction.

Bladder DESs are commonly used to open and alleviate pain caused by obstructions of the ureter. Obstructions of the ureter are caused by ureteral stones, severe constipation, cancerous and noncancerous tumors, internal tissue growth, such as endometriosis in females, long-term swelling of the ureter wall, usually due to diseases such as tuberculosis or a parasite infection called schistosomiasis. A cannabinoid eluting bladder DES maybe used to treat pain, inflammation and cancer growth associated with bladder cancer and ureter duct obstruction.

Duodenal and pancreatic DESs are commonly used to open obstructions associated with advanced malignancies in the head of the pancreas, duodenal cancer or gastric cancer, as well as invasive cancers. A cannabinoid eluting duodenal DES may provide enhanced palliative treatment for pain relief.

Gastric DESs are commonly used to open obstructions associated with benign tumors as well as malignancies in the esophagus and the gastroduodenum. Benign tumors in the esophagus include benign stricture, esophageal perforation, esophagobronchial fistula, refractory esophageal, and variceal bleeding. Benign tumors in the gastroduodenum include benign stricture. Malignant tumors of the esophagus include esophageal cancer, gastric cardia cancer, and extrinsic tumor invasion. Malignant tumors of the gastroduodenum include gastric cancer, duodenal cancer and pancreatic cancer.

Methods for Treatment:

Some embodiments relate to methods for treatment of disease, comprising administering to a patient in need of treatment, a DES or an IUD comprising a cannabinoid. The disease may be cancer. The cancer may be selected from the group consisting of: endometrial cancer, uterine sarcoma, cervical cancer, and ovarian cancer. The disease may be a disease in which a patient has an increased risk of cancer due to a genetic mutation. The disease in which a patient has an increased risk of cancer due to a genetic mutation may be Lynch syndrome. According to an embodiment, a patient having an increased risk of cancer is administered an IUD comprising a cannabinoid, before the appearance of a symptom of cancer.

The disease treated using an IUD may be selected from the group consisting of: depression, post-partum depression, post-partum psychosis, PTSD, bipolar disorder, anxiety, an eating disorder, fibromyalgia, PMS, and menopause related symptoms.

According to an embodiment the disease treated using an IUD may be selected from the group consisting of: abnormal pap smears, abnormal uterine bleeding, endometriosis, fibroids, menopause, ovarian masses, pelvic pain, polycystic ovarian syndrome, a sexually transmitted disease, an infection of the urinary tract and vaginitis. The sexually transmitted disease may be selected from the group consisting of chlamydia, genital herpes, gonorrhea, HIV/AIDS, Human papillomavirus, syphilis, and trichomoniasis.

According to an embodiment, the disease treated using a DES is selected from the group consisting of heart disease, coronary artery disease, angina, acute myocardial infarction, gallstones, injury, congenital malformations and biliary system related malignancies, bile duct cancer, pancreatic cancer, ureteral stones, severe constipation, cancerous or noncancerous tumors, internal tissue growth, endometriosis in females, swelling of the ureter wall, tuberculosis, schistosomiasis, obstruction of urine flow, duodenal cancer, gastric cancer, malignancies of the esophagus, benign tumors of the esophagus, benign stricture, esophageal perforation, esophagobronchial fistula, refractory esophageal and variceal bleeding, gastric cardia cancer, and extrinsic tumor invasion.

According to an embodiment, an IUD or DES is used to treat pain, inflammation or cancer growth associated with one of the aforementioned diseases or conditions.

According to an embodiment, the device, whether it is an IUD or a DES, upon insertion into a lumen of a patient in need thereof, administers to the patient a dose comprising a cannabinoid for a period of up to 3 months to up to 5 years. Optionally, the period is 3 months, 6 months, 1 year or 5 years.

According to an embodiment, the device, upon insertion into a lumen of a patient in need thereof, administers to the patient a dose of cannabinoid of between 0.5 microgram (μg) per day and 10 milligrams (mg) per day. Optionally, the dose is between 10 μg and 600 μg per day. Optionally the dose is between 50 μg and 500 μg per day.

The treatment of a disease comprising administering to a patient in need of treatment, an IUD or DES comprising a cannabinoid may be performed as a monotherapy. Alternatively, the treatment may be performed in combination with another agent. The other agent may be selected from the group consisting of: a hormone, a chemotherapeutic agent, an immunotherapeutic agent and a biological agent. The treatment may be performed in combination with radiation treatment.

The other agent may be an antineoplastic drug, an anti-inflammatory drug, an antibiotic an immunomodulator, an antiproliferative drug, a migration inhibitor, an ECM modulator, and an endothelialization factor. The agent may be selected from the group consisting of: sirolimus, tacrolimus, everolimus, leflunomide, M-prednisolone, dexamethasone, interferon, mycophenolic acid, mizoribine, cyclosporine, tranilast, paclitaxel, actinomycin, methotrexate, angiopeptin, cincristine, mitomycine, a statin, 2-chloro-deoxyadenosine, batimastat, halofunginone, probucol, VEGF and estradiol.

The following examples are provided to illustrate certain particular features and/or embodiments. These examples should not be construed to limit the disclosure to the particular features or embodiments described.

### EXAMPLES

### Example 1

IUD and DES comprising PLGA Biodegradable Polymer Matrix

Biodegradable polymer matrix can be manufactured using the excipients and active ingredients according to Table 1, below.

TABLE 1

| | | Range | Amount (mg) |
|---|---|---|---|
| Ingredient | Type of Ingredient | (w/w) | in IUD |
| CBD | Cannabinoid, active | 30-50 | 100 |
| PLGA | Polymer | 30-50 | 85 |
| Vitamin E TPGS | Nonionic Surfactant | 5-10 | 10 |
| Chitosan | Mucoadhesive agent | 1-10 | 5 |
| Total | — | 100 | 200 |

The CBD is dissolved in ethanol, and then the chitosan is added to the solution. PLGA copolymer is melted with Vitamin E TPGS at 80° C. The molten polymer is mixed with the organic solution containing the CBD and chitosan until a homogeneous mixture is obtained. In case any of the ingredients is not completely dissolved, small amounts of dichloromethane or acetone are added.

Solvents are evaporated and the mixture is cooled to room temperature and poured into a mold to obtain solidification to the desired dimensions of the biodegradable polymer matrix in the form of rods or cylinders to be inserted in the IUD reservoir, or may be formed as a coating to coat a DES or a portion thereof using any of the currently available stent coating techniques such as dip coating, spray coating, electro-treated coating, or plasma-treated coating.

Dip coating is a technique involving submerging the stent in a solution of typically drug and/or polymer in a solvent. The stent is then left to dry, allowing for evaporation, in the air or an oven.

Spray-coating includes ultrasonic atomization, electro-hydrodynamic jetting, and air-brush spray coating. These techniques use apparatuses that spray polymer and drug solutions (using various solvents) onto a stent, enabling consistent deposit of a uniform drug release layer(s) onto the stent surface.

Electro-treated coating incorporates electrical stimulus into the stent coating techniques to assist in drug/polymer deposition onto the stent surface or to increase polymerization on an already deposited drug release layer.

Plasma-treated coating technique involves exposing the base metal or polymer coated stent surface to a gaseous plasma beam for varying lengths of time in order to strengthen the chemical bonds in the drug release layer via polymer cross-linking.

### Example 2

IUD and DES comprising PCL Biodegradable Polymer Matrix

Compositions comprising the ingredients in Table 2 may be formed using the general method described in example 1.

TABLE 2

| Ingredient | Amount (mg) in IUD |
|---|---|
| CBD | 100 |
| PCL | 85 |
| Vitamin E TPGS | 10 |
| Chitosan | 5 |
| Total | 200 |

### Example 3

IUD and DES comprising PLGA Biodegradable Polymer Matrix containing 2 Cannabinoids

Compositions comprising the ingredients in Table 3 may be formed using the general method described in example 1.

TABLE 3

| Ingredient | Amount (mg) in IUD |
|---|---|
| CBD | 75 |
| THC | 25 |
| PLGA (50:50) | 85 |
| Vitamin E TPGS | 10 |
| Chitosan | 5 |
| Total | 200 |

### Example 4

IUD and DES comprising PLGA/PCL Biodegradable Polymer Matrix Composite

Compositions comprising the ingredients in Table 4 may be formed using the general method described in example 1.

TABLE 4

| Ingredient | Amount (mg) in IUD |
|---|---|
| CBD | 100 |
| PCL | 50 |
| PLGA (70:30) | 40 |
| Vitamin E TPGS | 5 |
| Chitosan | 5 |
| Total | 200 |

### Example 5

IUD and DES comprising PLGA Biodegradable Polymer Matrix of a Polylactide: Polyglycolide ratio of 85:15

Compositions comprising the ingredients in Table 5 may be formed using the general method described in example 1.

TABLE 5

| Ingredient | Amount (mg) in IUD |
|---|---|
| CBD | 100 |
| PLGA (85:15) | 90 |
| Vitamin E TPGS | 5 |
| Chitosan | 5 |
| Total | 200 |

### Example 6

IUD and DES comprising PLGA Biodegradable Polymer Matrix of a Polylactide: Polyglycolide ratio of 82:18

Compositions comprising the ingredients in Table 6 may be formed using the general method described in example 1.

TABLE 6

| Ingredient | Amount (mg) in IUD |
|---|---|
| CBD | 100 |
| PLGA (85:15) | 80 |
| Vitamin E TPGS | 10 |
| Chitosan | 10 |
| Total | 200 |

### Example 7

IUD and DES comprising PLGA Biodegradable Polymer Matrix with Cremophor RH40 as Surfactant

Compositions comprising the ingredients in Table 7 may be formed using the general method described in example 1.

TABLE 7

| Ingredient | Amount (mg) in IUD |
|---|---|
| CBD | 100 |
| PLGA (70:30) | 85 |
| Cremophor RH40 | 10 |
| Chitosan | 5 |
| Total | 200 |

### Example 8

IUD and DES comprising PLGA Biodegradable Polymer Matrix containing Penetration Enhancer

Compositions comprising the ingredients in Table 8 may be formed using the general method described in example 1.

TABLE 8

| Ingredient | Amount (mg) in IUD |
|---|---|
| CBD | 100 |
| PLGA (70:30) | 85 |
| Cremophor RH40 | 5 |
| Transcutol | 5 |
| Chitosan | 5 |
| Total | 200 |

### Example 9

Extrusion of Rods and Fibers Comprising CBD

Compositions were prepared in rod and fiber form using a number of compositions, according to Table 9, listed in weight percent:

TABLE 9

| | | PLGA | | | |
|---|---|---|---|---|---|
| Batch | CBD % | (75:25) % | PCL % | EVA % | Additive % |
| 1 | 30 | 70 | 0 | 0 | 0 |
| 2 | 30 | 65 | 0 | 0 | 5 (Kolliphor HS 15) |
| 3 | 30 | 0 | 70 | 0 | 0 |
| 4 | 30 | 0 | 65 | 0 | 5 (Kolliphor P188) |
| 5 | 30 | 0 | 0 | 70 | 0 |
| 6 | 30 | 0 | 0 | 65 | 5 (Kolliphor TPGS) |

In all of the compositions, CBD was introduced at 30% by weight. EVA is Ethylene-vinyl acetate (EVA), produced by Aldrich. The vinyl acetate content in the EVA was 8% by weight. Kolliphor HS 15 is a non-ionic solubilizer and emulsifier, also known as Macrogol-15-Hydroxystearate, obtained from Sigma. Kolliphor P188 (Micro) is a plasticizing agent, solubilizer and emulsifier, known as Poloxamer 188, obtained from Sigma. Kolliphor TPGS is a solubilizer and emulsifier known as Vitamin E Polyethylene Glycol Succinate and obtained from Sigma.

Compositions were formed using a twin-screw extruder, using the following process. Materials were kept into separate jars after weighing and were fed manually to the extruder. Polymer—drug—Polymer was the sequencing for the loading materials into the extruder. Temperature of the extruder zones were selected per melting temperature of the polymer to avoid any high torque issues. A chiller was utilized to avoid any melting of the polymer in a feed throat which can results into feeding issues. After the feeding, the materials were mixed in the extruder for about 10 mins followed by extrusion.

Each batch size was about 7 grams and the extrusion temperature was between 53° C. and 110° C., with the chiller set point being 20° C.

Characterization of the Resultant Formulations

In vitro release profiles were carried out to determine the release profiles of the cannabinoid in the biodegradable polymer matrix formed in the preceding examples, as a function of time using the following procedure. Triplicate sample from each batch of rod/fiber was weighed (about 3 mg) into a tube and added to 13 ml of 0.1% Tween 80 (polysorbate 80) in water. The sample tube was placed into a shaker bath set to 37° C. and 120 revolutions per minute. At the designated time points, each sample was removed from the bath and 1 ml of sample fluid was removed and analyzed for cannabinoid content by HPLC. 1 ml of fresh 0.1% Tween 80 in water solution was added for replacement fluid.

The cumulative release profiles were determined after 24 and 48 hours for batches 1 through 6, for rods and fibers, are shown in table 10.

TABLE 10

| | |
|---|---|
| | IVR |
| Content % | Cumulative release (%) |

| Batch | Shape | (CBD) | | 24 hrs | 48 hrs |
|---|---|---|---|---|---|
| 1 | Rods | 30.41 | Average | 1.07 | 1.65 |
| | | | SD | 0.01 | 0.04 |
| | Fibers | 28.91 | Average | 1.53 | 2.16 |
| | | | SD | 0.10 | 0.06 |
| 2 | Rods | 34.3 | Average | 2.73 | 3.41 |
| | | | SD | 0.10 | 0.14 |
| | Fibers | 32.3 | Average | 3.51 | 4.48 |
| | | | SD | 0.03 | 0.08 |
| 3 | Rods | 31.1 | Average | 20.23 | 27.48 |
| | | | SD | 0.22 | 2.70 |
| | Fibers | 30.9 | Average | 44.09 | 45.56 |
| | | | SD | 4.11 | 1.13 |
| 4 | Rods | 29.7 | Average | 27.44 | 33.61 |
| | | | SD | 5.31 | 5.70 |
| | Fibers | 29.3 | Average | 53.38 | 51.96 |
| | | | SD | 0.90 | 2.07 |
| 5 | Rods | 26.5 | Average | 75.79 | 72.88 |
| | | | SD | 0.93 | 1.18 |
| | Fibers | 29.4 | Average | 71.51 | 68.64 |
| | | | SD | 0.79 | 1.57 |
| 6 | Rods | 28.9 | Average | 59.86 | 66.93 |
| | | | SD | 0.62 | 0.65 |

Discussion:

High cannabinoid content (close to 30%) was present after extrusion, indicating that the process of extrusion of the compositions did not negatively impact the cannabinoid content.

Release profiles of the compositions varied depending upon composition. Rather rapid release was found with compositions based on EVA and PCL, whereas slow release, of less than 5% over 48 hours was seen with compositions based on PLGA 75:25. PLGA, with or without additional additives, can be considered a useful excipient for preparing compositions for sustained release within a human body for a period of one month or more.

The fibers and rods described in the above examples may be suitable to be introduced into a device when inserted into a lumen of a patient, releases cannabinoid for an extended period of time, preferably a month or more.

Physical and chemical stability follow up of the biodegradable polymer matrix formed in the examples may be performed at room and accelerated stress storage conditions (40° C.) by testing visual appearance, drug loading and drug degradation by suitable chromatographic methods (HPLC, GC), and DSC thermograms.

According to an embodiment, described herein is a device comprising a composition, the composition comprising at least one cannabinoid, wherein, when inserted into a lumen of a patient, releases cannabinoid for a period of at least 1 month. Optionally, the device is in the form of an intrauterine device, optionally a drug-eluting stent (DES). Optionally, the device comprises a biodegradable polymer matrix comprising a cannabinoid. Optionally, the biodegradable polymer matrix is contained within a reservoir of the device. Optionally, the biodegradable polymer matrix further comprises a surfactant. Optionally, the cannabinoid is selected from the group consisting of: cannabidiol, tetrahydrocannabinol, cannabigerol (CBG), cannabigerolic acid (CBGA), cannabigerol monomethyl ether (CBGM), cannabichromene (CBC), cannabichromanone (CBCN), cannabichromenic acid (CBCA), cannabivarichromene (CBCV), cannabichromevarinic acid (CBCVA), isotetrahydrocannabinol (iso-THC), cannabinol (CBN), cannabinolic acid (CBNA), cannabinol methyl ether (CBNM), cannabinol C4 (CBN-C4), cannabinol C2 (CBN-C2), cannabinol C1 (CBN-C1), cannabinodiol (CBND), cannabielsoin (CBE), cannabielsoic acid A (CBEA-A), Cannabielsoic acid B (CBEA-B), cannabicyclol (CBL), cannabicycloic acid (CBLA), cannabicyclovarin (CBLV), cannabitriol (CBT), cannabitriolvarin (CBTV), ethoxy-cannabitriolvarin (CBTVE), cannabivarin (CBV), cannabinodivarin (CBVD), tetrahydrocannabivarin (THCV), cannabidivarin (CBDV), cannabigerovarin (CBGV), cannabigerovarinic acid (CBGVA), cannabifuran (CBF), dehydrocannabifuran DCBF, and cannabirispol (CBR). Optionally, the cannabinoid is selected from the group consisting of: cannabidiol and tetrahydrocannabinol. Optionally, he biodegradable polymer matrix comprises cannabinoid in an amount between 2% and 50% by weight. Optionally, the biodegradable polymer matrix comprises cannabinoid in an amount between 20% and 40% by weight. Optionally, the biodegradable polymer matrix comprises cannabinoid in an amount of 30% by weight. Optionally, the device comprises between 1 mg and 100 mg of total cannabinoid. Optionally, the biodegradable polymer matrix comprises PLGA or PCL. Optionally, the biodegradable polymer matrix comprises PLGA. Optionally, the biodegradable polymer matrix comprises a surfactant. Optionally, the surfactant comprises polyoxyl 40 hydrogenated castor oil. Optionally, the surfactant comprises tocopheryl polyethylene glycol succinate, Macrogol-15-Hydroxystearate, or Poloxamer 188. Optionally, the surfactant is present in an amount between 4% and 6% by weight. Optionally, the biodegradable polymer matrix comprises a mucoadhesive agent. Optionally, the mucoadhesive agent is chitosan or a chitosan derivative. Optionally, upon introduction into a lumen of a human, the device releases between 0.5 microgram (μg) per day and 10 milligrams (mg) per day of cannabinoid. Optionally, upon introduction into a lumen of a human, the device releases between 10 μg per day and 600 μg per day of cannabinoid. Optionally, upon introduction into the uterus of a human, the device releases between 50 μg per day and 500 μg per day of cannabinoid.

According to a further embodiment, described herein is a method for treatment of a disease comprising administering to a patient in need thereof a device according to any one of the previous claims.

According to a further embodiment, described herein is a device according as described above for use in treatment of a disease comprising administering to a patient in need thereof. Optionally, the disease is cancer. Optionally, the disease is Lynch syndrome. Optionally, the disease is selected from the group consisting of: depression, post-partum depression, post-partum psychosis, PTSD, bipolar disorder, anxiety, an eating disorder, fibromyalgia, PMS, and menopause related symptoms. Optionally, the cancer is selected from the group consisting of: endometrial cancer, uterine sarcoma, cervical cancer, ovarian cancer, bile duct cancer, pancreatic cancer, duodenal cancer, gastric cancer, esophageal cancer, and gastric cardia cancer. Optionally, the disease is selected from the group consisting of: abnormal pap smears, abnormal uterine bleeding, endometriosis, fibroids, menopause, ovarian masses, pelvic pain, polycystic ovarian syndrome, a sexually transmitted disease, an infection of the urinary tract and vaginitis. Optionally, the sexually transmitted disease is selected from the group consisting of chlamydia, genital herpes, gonorrhea, HIV/AIDS, Human papillomavirus, syphilis, and trichomoniasis.

Optionally, the device is an intrauterine device administered to the uterus of a patient. Optionally, the disease is selected from the group consisting of: heart disease, coronary artery disease, angina, acute myocardial infarction, gallstones, injury, congenital malformations and biliary system related malignancies, ureteral stones, severe constipation, noncancerous tumors, internal tissue growth, endometriosis in females, swelling of the ureter wall, tuberculosis, schistosomiasis, obstruction of urine flow, benign tumors of the esophagus, benign stricture, esophageal perforation, esophagobronchial fistula, refractory esophageal and variceal bleeding, and extrinsic tumor invasion. Optionally, the device is a DES. Optionally, the device is used to treat pain or inflammation. Optionally, the amount of cannabinoid administered per day is 50 μg per day and 500 μg. Optionally, the device is administered to a lumen of a patient. Optionally, the lumen is a uterus, an artery, a biliary duct, a ureter, an intestine, a duodenum, a pancreas or a colon.

A further embodiment described herein is a method for manufacture of a device described above comprising inserting a biodegradable polymer matrix into a reservoir of the device. Optionally, the method further comprises forming a biodegradable polymer matrix by admixing a cannabinoid, a polymer, a surfactant and a mucoadhesive agent. Optionally, admixing is performed when heating the cannabinoid, polymer, surfactant and mucoadhesive agent.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

1. An intrauterine device comprising a composition, the composition comprising a biodegradable polymer matrix and at least one cannabinoid, wherein, when inserted into a uterus of a patient, releases cannabinoid for a period of at least 1 month.

2-4. (canceled)

5. The device according to claim 1 wherein the biodegradable polymer matrix is contained within a reservoir of the device.

6. The device according to claim 1, wherein the biodegradable polymer matrix further comprises a surfactant.

7. The device according to claim 1 wherein the cannabinoid is selected from the group consisting of: cannabidiol, tetrahydrocannabinol, cannabigerol (CBG), cannabigerolic acid (CBGA), cannabigerol monomethyl ether (CBGM), cannabichromene (CBC), cannabichromanone (CBCN), cannabichromenic acid (CBCA), cannabivarichromene (CBCV), cannabichromevarinic acid (CBCVA), isotetrahydrocannabinol (iso-THC), cannabinol (CBN), cannabinolic acid (CBNA), cannabinol methyl ether (CBNM), cannabinol C₄ (CBN-C₄), cannabinol C₂ (CBN-C₂), cannabinol C₁ (CBN-C₁), cannabinodiol (CBND), cannabielsoin (CBE), cannabielsoic acid A (CBEA-A), Cannabielsoic acid B (CBEA-B), cannabicyclol (CBL), cannabicycloic acid (CBLA), cannabicyclovarin (CBLV), cannabitriol (CBT), cannabitriolvarin (CBTV), ethoxy-cannabitriolvarin (CBTVE), cannabivarin (CBV), cannabinodivarin (CBVD), tetrahydrocannabivarin (THCV), cannabidivarin (CBDV), cannabigerovarin (CBGV), cannabigerovarinic acid (CBGVA), cannabifuran (CBF), dehydrocannabifuran DCBF, and cannabirispol (CBR).

8. The device according to claim 7 wherein the cannabinoid is selected from the group consisting of: cannabidiol and tetrahydrocannabinol.

9. (canceled)

10. The device according to claim 1 wherein the biodegradable polymer matrix comprises cannabinoid in an amount between 20% and 40% by weight.

11. (canceled)

12. The device according to claim 1, comprising between 1 mg and 100 mg of total cannabinoid.

13. The device according to claim 1 wherein the biodegradable polymer matrix comprises PLGA or PCL.

14. (canceled)

15. The device according to claim 1 wherein the biodegradable polymer matrix comprises a surfactant.

16. The device according to claim 15 wherein the surfactant comprises polyoxyl 40 hydrogenated castor oil, tocopheryl polyethylene glycol succinate, Macrogol-15-Hydroxystearate, or Poloxamer 188.

17-18. (canceled)

19. The device according to claim 1 wherein the biodegradable polymer matrix comprises a mucoadhesive agent.

20. The device according to claim 19 wherein the mucoadhesive agent is chitosan or a chitosan derivative.

21. The device according to claim 1 wherein, upon introduction into a lumen of a human, releases between 0.5 microgram (μg) per day and 10 milligrams (mg) per day of cannabinoid.

22. The device according to claim 21 wherein, upon introduction into a lumen of a human, releases between 10 μg per day and 600 μg per day of cannabinoid.

23. The device according to claim 22 wherein, upon introduction into the uterus of a human, releases between 50 μg per day and 500 μg per day of cannabinoid.

24. A method for treatment of a disease comprising administering to a patient in need thereof a device according to claim 1.

25. The method according to claim 24 wherein the disease is cancer.

26. The method according to claim 24 wherein the disease is Lynch syndrome.

27. The method according to claim 24 wherein the disease is selected from the group consisting of: depression, post-partum depression, post-partum psychosis, PTSD, bipolar disorder, anxiety, an eating disorder, fibromyalgia, PMS, menopause related symptoms, abnormal pap smears, abnormal uterine bleeding, endometriosis, fibroids, menopause, ovarian masses, pelvic pain, polycystic ovarian syndrome, a sexually transmitted disease, and an infection of the urinary tract and vaginitis.

28. The method according to claim 25 wherein the cancer is selected from the group consisting of: endometrial cancer, uterine sarcoma, cervical cancer, ovarian cancer, bile duct cancer, pancreatic cancer, duodenal cancer, gastric cancer, esophageal cancer, and gastric cardia cancer.

29-40. (canceled)
